(19) United States
(12) Patent Application Publication
(10) Pub. No.: US 2013/0261018 A1
(43) Pub. Date: Oct. 3, 2013

(54) INTERFERON GENE SIGNATURE AND METHODS OF USE THEREOF

(71) Applicants: Lawrence Pfeffer, Germantown, TN (US); Meiyun Fan, Lakeland, TN (US); Susan R. Pfeffer, Germantown, TN (US); Charles Handorf, Memphis, TN (US)

(72) Inventors: Lawrence Pfeffer, Germantown, TN (US); Meiyun Fan, Lakeland, TN (US); Susan R. Pfeffer, Germantown, TN (US); Charles Handorf, Memphis, TN (US)

(73) Assignee: University of Tennessee Research Foundation, Knoxville, TN (US)

(21) Appl. No.: 13/880,209

(22) PCT Filed: Oct. 13, 2011
(86) PCT No.: PCT/US11/56063

§ 371 Date: Apr. 23, 2013

Related U.S. Application Data
(60) Provisional application No. 61/394,803, filed on Oct. 20, 2010.

(51) Int. Cl. G01N 33/68

(52) U.S. Cl. CPC G01N 33/6866
USPC 506/9; 435/6.12; 436/501; 435/6.11; 435/7.92; 506/16; 506/18

(57) ABSTRACT

The present invention is a method and kit for determining responsiveness of a subject to treatment with a Type I interferon based upon the expression of gene signatures shown to be stimulated by interferon. A method for testing the propensity of a test compound to induce a Type I interferon response is also provided.

This patent application claims the benefit of priority from U.S. Provisional Application Ser. No. 61/394,803 filed Oct. 20, 2010, the content of which is incorporated herein by reference in its entirety.

### BACKGROUND OF THE INVENTION

Interferons (IFNs) are a well-known family of cytokines secreted by a large variety of eukaryotic cells upon exposure to various stimuli. The interferons have been classified by their chemical and biological characteristics into four groups: IFN-alpha (leukocytes), IFN-beta (fibroblasts), IFN-gamma (lymphocytes), and IFN-omega (leukocytes). IFN-alpha and beta are known as Type I interferons, whereas IFN-gamma is known as a Type II or immune interferon.

By interacting with specific receptors, IFNs activate Signal Transducer and Activator of Transcription (STAT) complexes. STAT activation initiates the classical Janus kinase-STAT (JAK-STAT) signaling pathway as well as several other signaling cascades (Platanias (2005) Nature Rev. Immunol. 5(5):375-386). IFNs also activate members of the nuclear factor-kappa B (NF-κB) transcription factor family that regulate many genes involved in cell survival and inflammatory responses (Du (2007) J. Cellular Biochemistry 102 (5):1087-1094).

IFNs exhibit anti-viral, immunoregulatory, and anti-proliferative activity. In this respect, the clinical potential of interferons has been recognized. For example, IFN beta-1a and IFN beta-1b are used to treat and control multiple sclerosis (Paolicelli, et al. (2009) Biologics: Targets & Therapy 3:369-76). Interferon therapy is also used (in combination with chemotherapy and radiation) as a treatment for many cancers (Goldstein & Laszlo (1988) CA Cancer J. Clin. 38(5):258-77). Both hepatitis B and hepatitis C are treated with IFN-alpha, often in combination with other antiviral drugs (Cooksley (2004) MedGenMed 6(1):16; Shepherd, et al. (2000) Health Technol. Assess. 4(33):1-67).

Efficacy of IFN treatment, based upon marker detection, has been suggested. For example, the detection of a nucleotide variation at position 937 of the HCV-1a NS5A gene (U.S. Pat. No. 7,504,217), or expression of a short form of interferon-gamma-inducible protein 10 (sIP-10)(U.S. Pat. No. 7,674,578); HuIFRG 198 protein (US 20070226815); HuIFRG 68.1 protein (US 20040175803); HuIFRG 55.1 protein (US 20040170961); HuIFRG 70 protein and related proteins (US 20060275258; US 20050265967); HuIFRG 28-1 protein (US 20050176094); HuIFRG 15.4 protein (US 20030176341); HUIFRG46/ADIR (ATP dependent IFN responsive) protein (US 20050129657); HuIFRG-1, HuIFRG-2, HuIFRG-3 and HuIFRG-4 proteins (US 20060099174); or XAF-1 gene (US 20070218493) has been suggested for use in predicting an individual's response to interferon treatment. Similarly, a method for diagnosing a predisposition of a multiple sclerosis (MS) patient for responsiveness to a treatment of MS with interferon-alpha and/or interferon-beta by detecting particular nucleic acid sequence motifs has been described (20090186773). Furthermore, diagnostic methods for detecting the presence of a factor that prevents the biological effect of a type I (US 20100129788) and detecting the upregulation of ERP-70, PIN-1, MIG, LTS, IP-10, Lipocalin 1, SEC 63, surfeit 6, PDE2A, KIAA0212, NPIK-B, BAF53a, MEF2C, AADAC, TPM1 or SPARC (US 20070117145) for predicting responsiveness to treatment with interferon-alpha are also described. Moreover, gene polymorphisms in STAT3 gene, SSI3 gene, IL-4R gene, IRF2 gene, ICSBP gene, PTGS1 gene, PTGS2 gene, or TAP2 gene have been suggested for use in evaluating tumor suppression by IFN therapy in renal cell cancer (US 20080199859).

### SUMMARY OF THE INVENTION

The present invention is a method for determining responsiveness of a subject to treatment with a Type I interferon (IFN) by detecting in a sample from the subject the mRNA or protein expression of one or more IFN-stimulated genes regulated by Signal Transducer and Activator of Transcription 2 (STAT2), IFN-stimulated genes regulated by STAT2 and Nuclear factor-kappa-B (NF-κB), IFN-stimulated genes regulated by STAT1/1, STAT1/3 OR STAT3/3 dimers, or IFN-stimulated genes regulated by STAT1/1, STAT1/3 OR STAT3/3 dimers and NF-κB, wherein the sample is obtained from the subject following administration of a Type I IFN or the sample is treated with a Type I IFN in vitro prior to detecting mRNA or protein expression, and wherein said mRNA or protein expression is indicative of the responsiveness of the subject to treatment with the Type I IFN.

A method for testing the propensity of a test compound to induce a Type I IFN response in a test subject is also provided. This method involves administering an effective amount of a test compound to a test subject or sample from the test subject; and determining changes in mRNA or protein expression of one or more IFN-stimulated genes regulated by Signal Transducer and Activator of Transcription 2 (STAT2), IFN-stimulated genes regulated by STAT2 and Nuclear factor-kappa-B (NF-κB), the IFN-stimulated genes regulated by STAT1/1, STAT1/3 OR STAT3/3 dimers, or IFN-stimulated genes regulated by STAT1/1, STAT1/3 OR STAT3/3 dimers and NF-κB, wherein an increase in mRNA or protein expression is indicative of the propensity of the test compound to induce a Type I interferon response.

In one embodiment of the invention, the IFN-stimulated genes regulated by STAT2 are selected from the group of Dhx58, Eif2ak2, IFI16, IFI6, Irf2, Mx1, OAS1, PLSCR1, Pm1, RARRES3, Rsad2, Socs1, Stat2, Tap1, Tlr2, Tlr3 and Tnfsf10. In another embodiment, the IFN-stimulated genes regulated by STAT2 and NF-κB are selected from the group of ANGPT2, Cc15, Ddx58, Gbp1, IFIH1, Ifnb1, Il6, Irf5, Irf7, Isg20, IRF9 and Stat1. In a further embodiment, the IFN-stimulated genes regulated by STAT1/1, STAT1/3 or STAT3/3 dimers is selected from the group of Casp4, Tnfrsflob, VEGFC and VISA. In yet another embodiment, the IFN-stimulated genes regulated by STAT1/l, STAT1/3 or STAT3/3 dimers and NF-κB are selected from the group of Gadd45b, Gadd45g, HIF3A, Irfl, Myd88 and XAF1. In yet further embodiment, all IFN-stimulated genes are detected. In a further embodiment, reagents for measuring the mRNA or protein expression of these genes are provided in the form of a kit.

### DETAILED DESCRIPTION OF THE INVENTION

Studies have described the beneficial effects of Type I interferon (IFN alpha and IFN beta) in the treatment of a wide variety of diseases. IFNs have been used therapeutically in multiple sclerosis (MS), chronic hepatitis C (HCV), chronic hepatitis B (HBV) and certain types of solid and hematological malignancies. It is therefore highly desirable to find appropriate biological markers for monitoring responsiveness and clinical efficacy of treatment with a Type I IFN.

Binding of IFN to its receptor on the surface of the cellular membrane induces a signal cascade resulting in the activation of transcription factors and synthesis of different proteins, so-called interferon-induced proteins. A panel of genes induced by type I interferons have now been identified and stratified into different signaling pathways. The activation status of these signaling pathways is altered in a number of disease states including viral infections such as Hepatitis C and in various forms of cancer. The level of induction of these genes can now be used to determine interferon responsiveness. As such, the gene signatures described herein are of use in identifying patients that are most likely to respond or not respond to interferon therapy; for monitoring the clinical efficacy of treatment with a Type I IFNs; and identifying test compounds that induce the gene signatures.

To identify the gene signatures of this invention, microarray analysis was performed on various human and mouse cells treated in the presence and absence of interferon to identify interferon-stimulated genes (ISGs). The results were compared to a public database of ISGs. The ISGs were than stratified into genes that were most commonly induced by interferon and had putative binding sites (and strong binding scores) for STAT2 (ISRE), STAT3/STAT1 (SIE), and NF-κB. The ISGs were verified by quantitative real-time PCR as being strongly interferon induced and subsequently grouped into the categories of: IFN-stimulated genes regulated by STAT2 (ISRE)(Table 1); IFN-stimulated genes regulated by NF-κB (Table 2); IFN-stimulated genes regulated by STAT1/1, STAT1/3 or STAT3/3 dimers (Table 3); and IFN-stimulated genes regulated by STAT1/1, STAT1/3 or STAT3/3 dimers and NF-κB (Table 4).

TABLE 1

| | | Accession No. | |
|---|---|---|---|
| Gene | Description | Nucleotide | Protein |
| Dhx58 | DEXH (Asp-Glu-X- | NM_024119 | NP_077024 |
| | His) box polypeptide | | |
| | 58 | | |
| Eif2ak2 | Eukaryotic | NM_001135651 | NP_001129123 |
| | translation initiation | NM_001135652 | NP_001129124 |
| | factor 2-alpha | NM_002759 | NP_002750 |
| | kinase 2 | | |
| IFI16 | Interferon, gamma- | NM_005531 | NP_005522 |
| | inducible protein 16 | | |
| IFI6 | Interferon, alpha- | NM_002038 | NP_002029 |
| | inducible protein 6 | NM_022872 | NP_075010 |
| | | NM_022873 | NP_075011 |
| Irf2 | Interferon regulatory | NM_002199 | NP_002190 |
| | factor 2 | | |
| Mxl | Interferon-induced | NM_002463 | NP_002454 |
| | GTP-binding protein | | |
| | Mx1 | | |
| OAS1 | 2′,5′-Oligoadenylate | NM_001032409 | NP_001027581 |
| | synthetase 1, 40/46 | NM_002534 | NP_002525 |
| | kDa | NM_016816 | NP_058132 |
| PLSCR1 | Phospholipid | NM_021105 | NP_066928 |
| | scramblase 1 | | |
| Pml | Promyelocytic | NM_002675 | NP_002666 |
| | leukemia | NM_033238 | NP_150241 |
| | | NM_033239 | NP_150242 |
| | | NM_033240 | NP_150243 |
| | | NM_033244 | NP_150247 |
| | | NM_033246 | NP_150249 |
| | | NM_033247 | NP_150250 |
| | | NM_033249 | NP_150252 |
| | | NM_033250 | NP_150253 |
| RARRES3 | Retinoic acid receptor | NM_004585 | NP_004576 |
| | responder (tazarotene | | |
| | induced) 3 | | |
| Rsad2 | Radical S-adenosyl | NM_080657 | NP_542388 |
| | methionine domain | | |
| | containing 2 | | |
| Socs1 | Suppressor of cytokine | NM_003745 | NP_003736 |
| | signaling 1 | | |
| Stat2 | Signal transducer and | NM_005419 | NP_005410 |
| | activator of | NM_198332 | NP_938146 |
| | transcription 2 | | |
| Tap1 | Transporter 1, ATP- | NM_000593 | NP_000584 |
| | binding cassette, sub- | | |
| | family B | | |
| Tlr2 | Toll-like receptor 2 | NM_003264 | NP_003255 |
| Tlr3 | Toll-like receptor 3 | NM_003265 | NP_003256 |
| Tnfsf10 | Tumor necrosis factor | NM_001190942 | NP_001177871 |
| | (ligand) superfamily, | NM_001190943 | NP_001177872 |
| | member 10 | NM_003810 | NP_003801 |

TABLE 2

| | | Accession No. | |
|---|---|---|---|
| Gene | Description | Nucleotide | Protein |
| ANGPT2 | Angiopoietin 2 | NM_001118887 | NP_001112359 |
| | | NM_001118888 | NP_001112360 |
| | | NM_001147 | NP_001138 |
| Ccl5 | Chemokine (C-C | NM_002985 | NP_002976 |
| | motif) ligand 5 | | |
| Ddx58 | DEAD box | NM_014314 | NP_055129 |
| | polypeptide 58 | | |
| Gbp1 | Guanylate binding | NM_002053 | NP_002044 |
| | protein 1, interferon- | | |
| | inducible, 67 kDa | | |
| IFIH1 | Interferon induced | NM_022168 | NP_071451 |
| | with helicase C | | |
| | domain 1 | | |
| Ifnb1 | Interferon, beta 1, | NM_002176 | NP_002167 |
| | fibroblast | | |
| Il6 | Interleukin 6 | NM_000600 | NP_000591 |
| | (interferon, beta 2) | | |
| Irf5 | Interferon regulatory | NM_001098627 | NP_001092097 |
| | factor 5 | NM_001098629 | NP_001092099 |
| | | NM_001098630 | NP_001092100 |
| | | NM_032643 | NP_116032 |
| Irf7 | Interferon regulatory | NM_001572 | NP_001563 |
| | factor 7 | NM_004029 | NP_004020 |
| | | NM_004031 | NP_004022 |
| Isg20 | Interferon stimulated | NM_002201 | NP_002192 |
| | exonuclease gene | | |
| | 20 kDa | | |
| IRF9 | Interferon regulatory | NM_006084 | NP_006075 |
| | factor 9 | | |
| Stat1 | Signal transducer and | NM_007315 | NP_009330 |
| | activator of | NM_139266 | NP_644671 |
| | transcription 1 | | |

TABLE 3

| | | Accession No. | |
|---|---|---|---|
| Gene | Description | Nucleotide | Protein |
| Casp4 | Caspase 4, apoptosis- | NM_001225 | NP_001216 |
| | related cysteine peptidase | NM_033306 | NP_150649 |
| Tnfrsf10b | Tumor necrosis factor | NM_003842 | NP_003833 |
| | receptor superfamily, | NM_147187 | NP_671716 |
| | member 10b | | |
| VEGFC | Vascular endothelial | NM_005429 | NP_005420 |
| | growth factor C | | |
| VISA | Virus-induced signaling | NM_020746 | NP_065797 |
| | adapter | | |

TABLE 4

| | | Accession No. | |
|---|---|---|---|
| Gene | Description | Nucleotide | Protein |
| Gadd45b | Growth arrest and | NM_015675 | NP_056490 |
| | DNA-damage- | | |
| | inducible, beta | | |
| Gadd45g | Growth arrest and | NM_006705 | NP_006696 |
| | DNA-damage- | | |
| | inducible, gamma | | |
| HIF3A | Hypoxia inducible | NM_022462 | NP_071907 |
| | factor 3, alpha | NM_152794 | NP_690007 |
| | subunit | NM_152795 | NP_690008 |
| Irf1 | Interferon regulatory | NM_002198 | NP_002189 |
| | factor 1 | | |
| Myd88 | Myeloid | NM_001172566 | NP_001166037 |
| | differentiation primary | NM_001172567 | NP_001166038 |
| | response gene (88) | NM_001172568 | NP_001166039 |
| | | NM_001172569 | NP_001166040 |
| | | NM_002468 | NP_002459 |
| XAF1 | X-linked inhibitor of | NM_017523 | NP_059993 |
| | apoptosis associated | NM_199139 | NP_954590 |
| | factor 1 | | |

Based upon the identified correlation, the interferon-stimulated genes of this invention are of use in methods for monitoring responses to Type I interferon or molecules that stimulate or induce Type I interferon signal transduction pathways, particularly in therapeutic applications. Accordingly, the present invention features a method for determining responsiveness of a subject to treatment with a Type I IFN, e.g., IFN-alpha treatment (such as IFN-alpha treatment) by detecting, in a sample from a subject, the mRNA or protein expression of one or more of the markers listed in Tables 1-4, wherein the sample is obtained from the subject following administration of a Type I IFN or the sample is treated with a Type I interferon in vitro prior to determining the mRNA or protein expression.

The phrase “responsiveness of a subject,” as used herein, refers to whether a subject responds positively (responder) or negatively (non-responder) to treatment with a Type I IFN. In this respect, “responders” are defined herein as subjects undergoing or about to undergo an IFN treatment and show or will show a good clinical response to this treatment, e.g., a decrease or amelioration of the signs or symptoms of the disease being treated. “Non responders” are defined herein as subjects undergoing or about to undergo an IFN treatment and receive or will receive little or no benefit from treatment with the interferon. In accordance with the present invention, responsiveness to IFN is determined in subjects who have yet to receive IFN by treating a sample from a subject with a Type I interferon in vitro.

The terms “Type I interferon” and “interferon” are used herein interchangeably and refer to the family of highly homologous species-specific proteins that inhibit viral replication and cellular proliferation and modulate immune response. Typical suitable interferons include, but are not limited to, recombinant interferon alpha-2b such as INTRON A interferon available from Schering Corporation, Kenilworth, N.J.; recombinant interferon alpha-2a such as ROFERON-A interferon available from Hoffmann-La Roche, Nutley, N.J.; recombinant interferon alpha-2C such as BEROFOR alpha 2 interferon available from Boehringer Ingelheim Pharmaceutical, Inc., Ridgefield, Conn.; a purified blend of natural alpha interferons such as SUMIFERON available from Sumitomo, Japan or as WELLFERON interferon alpha-n1 (INS) available from the Glaxo-Wellcome Ltd., London, GB; or a consensus alpha interferon such as those described in U.S. Pat. Nos. 4,897,471 and 4,695,623; and the specific product available from Amgen, Inc., Newbury Park, Calif.; or interferon alpha-n3 a mixture of natural alpha interferons made by Interferon Sciences and available from the Purdue Frederick Co., Norwalk, Conn. under the ALFERON Tradename. Type I IFN also includes pegylated interferon alpha including, but are not limited to, PEGASYS (peginterferon alpha-2a) and PEG-INTRON (peginterferon alpha-2b). Preferably, the Type I IFN for testing responsiveness will be the Type I interferon selected for treatment. It can be administered by an appropriate treatment route and at an appropriate treatment dose.

Subjects in accordance with the instant methods include human and non-human animals with immune system-related disorders such as viral infection, parasitic infection, bacterial infection, cancer, autoimmune disease, multiple sclerosis, lymphoma or allergy. Exemplary conditions in which interferon treatment is employed include, but are not limited to, cell proliferation disorders, in particular cancer (e.g., hairy cell leukemia, Kaposi's sarcoma, chronic myelogenous leukemia, multiple myeloma, basal cell carcinoma and malignant melanoma, ovarian cancer, cutaneous T cell lymphoma), and viral infections. Without limitation, treatment with interferon may be used to treat conditions which would benefit from inhibiting the replication of interferon-sensitive viruses. Viral infections of this type include hepatitis A, hepatitis B, hepatitis C, other non-A/non-B hepatitis, herpes virus, Epstein-Barr virus (EBV), cytomegalovirus (CMV), herpes simplex, human herpes virus type 6 (HHV-6), papilloma, poxvirus, picornavirus, adenovirus, rhinovirus, human T lymphotropic virus-Type I and 2 (HTLV-1/-2), human rotavirus, rabies, retroviruses including human immunodeficiency virus (HIV), encephalitis and respiratory viral infections.

The term “sample” or “biological sample” refers to a sample of tissue or fluid isolated from a subject, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, and organs obtained by routine methods. Also included are samples of in vitro cell culture constituents (including, but not limited to, conditioned medium resulting from the growth of cells in culture medium, putatively virally infected cells, recombinant cells, and cell components).

In carrying out the methods of the invention, the mRNA or protein expression of one or more of the markers listed in Tables 1-4 is detected. This step can include detecting the expression of one, two, three, four, five or more of the markers in Table 1, Table 2, Table 3, or Table 4; detecting the expression of all the markers in Table 1, Table 2, Table 3, or Table 4; detecting the expression of one, two, three, four, five or more markers from Table 1, Table 2, Table 3, and Table 4; or detecting the expression of all 39 markers from Tables 1-4. The combination of markers detected can be based on various factors including, for example, the disease or condition being treated, the Type I IFN being administered and/or the desired outcome of the treatment.

Numerous techniques for detecting mRNA or protein expression are known in the art and can all be used to practice the methods of the present invention. The particular method used to detect mRNA or protein expression the sequence variation is not a critical aspect of the invention. Although considerations of performance, cost, and convenience will make particular methods more desirable than others, it will be clear that any method that can detect the expression of an mRNA or protein listed in Tables 1-4 will provide the information needed to practice the invention. The techniques can be polynucleotide-based or protein-based. In either case, the techniques used must be sufficiently sensitive so as to accurately detect the presence and/or level of mRNA or protein expression.

Numerous polynucleotide-based methods are routinely used in the art for detecting, evaluating and quantifying mRNA expression. For example, levels of mRNA can be evaluated using well-known methods such as northern blot analysis (see, e.g., Sambrook and Russell (2001) In: Molecular Cloning, a Laboratory Manual, Cold Spring Harbor Laboratory; 3rd edition); oligonucleotide or cDNA fragment hybridization wherein the oligonucleotide or cDNA is configured in an array on a chip or wafer; RNase protection analysis; or RT-PCR.

Suitable primers, probes, or oligonucleotides useful for such detection methods can be generated by the skilled artisan from the sequences provided in Tables 1-4 or paralogs, orthologs or naturally occurring variants (e.g., allelic variants) thereof. The term primer, as defined herein, is meant to encompass any nucleic acid that is capable of priming the synthesis of a nascent nucleic acid in a template-dependent process. Typically, primers are oligonucleotides from ten to twenty-five base pairs in length, but longer sequences can be employed. Primers can be provided in double-stranded or single-stranded form. Probes are defined differently, although they can act as primers. Probes, while perhaps capable of priming, are designed for hybridizing to the target DNA or RNA and need not be used in an amplification process. In particular embodiments, the probes or primers are labeled with, for example, radioactive species (³²P, ¹⁴_{C,} ³⁵S, ³H, or other label) or a fluorophore (rhodamine, fluorescein). Depending on the application, the probes or primers can be used cold, i.e., unlabeled, and the RNA or cDNA molecules are labeled.

Various RT-PCR methodologies can be employed to evaluate the level of mRNA present in a sample. As clinical samples are of variable quantity and quality a relative quantitative RT-PCR reaction can be performed with an internal standard. The internal standard can be an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other assays can be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various samples must be carefully normalized for equal concentrations of amplifiable cDNAs. This consideration is very important since the assay measures absolute mRNA abundance. Absolute mRNA abundance can be used as a measure of differential gene expression in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

Specifically contemplated by the present invention are chip-based technologies. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see, e.g., Pease, et al. (1994) Proc. Natl. Acad. Sci. USA 91(11):5022-6; Fodor, et al. (1991) Science 251(4995):767-73).

Depending on the format, detection can be performed by visual means (e.g., ethidium bromide staining of a gel). Alternatively, detection can involve indirect identification of the product via chemiluminescence, radiolabel or fluorescent label or even via a system using electrical or thermal impulse signals (Bellus (1994) J. Macromol. Sci. Pure Appl. Chem. A311:1355-1376).

In an alternative embodiment, one or more IFN-stimulated proteins are detected in a sample. In general, the detection of a protein of Table 1-4 is carried out by immunoassays using antibodies which specifically bind to said proteins. Antibodies of use in the instant methods can be either polyclonal or monoclonal. Moreover, such antibodies can be natural or partially or wholly synthetically produced. All fragments or derivatives thereof which maintain the ability to specifically bind to the protein of interest are also included. The antibodies can be a member of any immunoglobulin class, including any of the human classes: IgG, IgM, IgA, IgD, and IgE.

Antibody fragments can be any derivative of an antibody which is less than full-length. In general, an antibody fragment retains at least a significant portion of the full-length antibody's specific binding ability. Examples of antibody fragments include, but are not limited to, Fab, Fab′, F(ab′)₂, scFv, Fv, diabody, or Fd fragments. The antibody fragment can be produced by any means. For instance, the antibody fragment can be enzymatically or chemically produced by fragmentation of an intact antibody or it can be recombinantly produced from a gene encoding the partial antibody sequence. The antibody fragment can optionally be a single-chain antibody fragment. Alternatively, the fragment can be multiple chains which are linked together, for instance, by disulfide linkages. The fragment can also optionally be a multi-molecular complex. A functional antibody fragment typically contains at least about 50 amino acids and more typically contains at least about 200 amino acids.

An antibody for use in the methods of the present invention can be generated using classical cloning and cell fusion techniques. Alternatively, antibodies of this invention can be produced by a phage display method. Methods of producing phage display antibodies are well-known in the art (e.g., Huse, et al. (1989) Science 246(4935):1275-81). As a further alternative, an antibody specific for a protein listed in Table 1-4 can be obtained from a commercial source. For the purposes of this invention, antibodies are used to evaluate the level or presence or absence of a marker via techniques such as ELISA, western blotting, or immunohistochemistry. A general method for detecting a marker includes contacting a sample with an antibody which specifically binds the marker, and detecting the presence of the antibody-antigen complex using any conventional immunoassay routinely used to detect and/or quantitate antigens (see, for example, Harlow and Lane (1988) Antibodies: A Laboratory Manual, Cold Spring Harbor Laboratory, New York). Such well-known immunoassays include antibody capture assays, antigen capture assays, and two-antibody sandwich assays.

After detecting the level, presence or absence of a transcript or protein present in a sample, the results seen in a given subject can be compared with a known standard or control. A known standard can be a statistically significant reference group of normal subjects or subjects that do or do not respond to interferon treatment to provide predictive information pertaining the subject from whom the sample was obtained. Alternatively, the level, presence or absence of a transcript or protein present in a sample can be compared with a sample from the same subject prior to treatment with interferon. In this respect, pre- and post-treatment levels of marker expression are compared to determine responsiveness of the subject to interferon treatment.

By determining responsiveness of a subject or efficacy of an IFN treatment, the instant methods find application in adjusting an IFN-therapy in a subject. For example, once a subject is identified as a responder, IFN therapy can be continued, modified (e.g., dosage increased or decreased) or initiated. In the case the subject is identified as a non responder, IFN treatment with a type I interferon can be modified (e.g., or administration of a different IFN), not initiated or discontinued.

Using the instant panel of markers, novel drugs or interferon mimetics that induce a Type I IFN response can also be identified. In this respect, the present invention further features a method for testing or determining the propensity of a test compound to induce a Type I IFN response. The method involves administering an effective amount of a test compound to a test subject or sample from a test subject and determining changes in mRNA or protein expression of one or more markers listed in Table 1-4, wherein an increase in mRNA or protein expression is indicative of the propensity of the test compound to induce a Type I interferon response.

For the purposes of the present invention, the term “test compound” refers in general to a compound to which a test subject or test sample is exposed. Typical test compounds will be small organic molecules, typically drugs and/or prospective pharmaceutical lead compounds, but can include proteins, peptides, polynucleotides, heterologous genes (in expression systems), plasmids, polynucleotide analogs, peptide analogs, lipids, carbohydrates, viruses, phage, parasites, and the like. Test compounds also include variants, analogs, and mimetics of a Type I interferon.

The term “test subject” refers to a human or non-human model capable of reacting to the presence of a test compound, wherein said test subject is typically a live non-human animal, or cell or tissue sample from an animal.

Changes in mRNA or protein expression of one or more markers listed in Table 1-4 can be determined as described herein. Protein or mRNA expression in response to the test compound can be compared to expression of the same in a subject or sample not receiving the test compound or expression in response to a “control compound,” which is known to induce a Type I interferon response (e.g., IFN alpha). The expression response can be weighted or scaled as necessary to normalize data, and can be reported as the absolute increase or decrease in expression (i.e., transcription or translation), the relative change (for example, the percentage change), the degree of change above a threshold level, and the like. The change in expression (or lack of change) of an mRNA or protein of Table 1-4 in response to administration of a test compound is indicative of the test compounds ability to induce a IFN response. In particular, an increase in expression of one, two, three or all of the mRNAs or proteins of Table 1-4 indicates that the test compound can induce a Type I IFN response. It is expected that test compounds shown to induce a Type I IFN response will be of use in the treatment of one or more of the diseases described herein.

Also provided are kits for use in practicing the instant methods. The term “kit” as used herein refers to any combination of reagents or apparatus that can be used to perform a method of the invention. In one embodiment, the present invention provides a kit including primer pairs specific to each of the nucleic acids listed in Table 1, Table 2, Table 3, or Table 4. In another embodiment, the present invention provides a kit including primer pairs specific to each of the nucleic acids listed in Table 1, Table 2, Table 3, and Table 4. Primer pairs for a combination of nucleic acids selected from Tables 1-4 is also contemplated. Preferably, each primer pair is capable of hybridizing to the mRNA or cDNA of the nucleic acids of Tables 1-4 and facilitating amplification of the same. Such primer pairs are routinely generated in the art and/or obtained from commercial sources (e.g., Origene or SABiosciences). Typically, the primer pair is capable of generating a 100-900 base pair amplicon, which can be detected by gel electrophoresis, probe hybridization, or other conventional methods for detecting nucleic acids. In this respect, the kit can also include probes designed to hybridize to an internal region of each amplicon of the nucleic acids listed in Tables 1-4.

Control primer pairs specific for a control gene, and a control probe designed to anneal to an internal region of the produced control amplicon can also be included in the kit. Examples of such controls include, but are not limited to ribosomal proteins such as RPLP0 (ribosomal protein, large, P0), glyceraldehyde-3-phosphate dehydrogenase, beta actin, MHC I (major histocompatibility complex I), cyclophilin, and 28S or 18S rRNAs (ribosomal RNAs).

In an alternative embodiment, a kit of the invention includes antibodies specific for each of the proteins listed in Table 1, Table 2, Table 3, or Table 4. In another embodiment, the present invention provides a kit including antibodies specific to each of the proteins listed in Table 1, Table 2, Table 3, and Table 4. Antibodies for a combination of proteins selected from Tables 1-4 is also contemplated. One or more control antibodies can also be included, which are specific for control proteins.

In some embodiments, the kit further includes one or more vessels suitable for accepting a sample. In one embodiment, a type I interferon (e.g., 10 and 100 IU/mL) is present inside the vessel. The type I interferon can be provided in said vessel in a liquid or lyophilized form. Alternatively, the type I interferon can be immobilized on part or all of the inside surface of said vessel. The inside wall of the vessel may be lined with a suitable coating enabling the type I interferon to be attached. In another embodiment, said type I interferon is immobilized on a solid support. The solid support may be attached to the inside of the vessel. Alternatively, the solid support may be free of the inside of the vessel. Examples of solid supports include, but are not limited to, chromatography matrix, magnetic beads.

In another embodiment, the vessel can include a stabilizing agent that inhibits cellular RNA degradation and/or gene induction. For example, the stabilizing agent can be that as found in a PAXGENE Blood RNA Tube. Alternatively, a quaternary amine surfactant can be used as a stabilizing agent. Suitable quaternary amine surfactants, able to stabilize RNA in biological samples, are described in U.S. Pat. No. 5,985,572, WO 94/18156 and WO 02/00599. One example of a quaternary amine which can be used in a kit of the present invention is tetradecyltrimethyl-ammonium oxalate. Alternatively, a cationic detergent such as CATRIMOX-14 can be used as stabilizing agent.

The kit can further include additional components for carrying out the method of the invention, such as RNA extraction solutions, purification columns and buffers and the like. The kit of the invention can also provide instructions for practicing the present invention. These instructions can be present in the kits in a variety of forms including printed information on a piece of paper, on the packaging of the kit, in a package insert, etc. Yet another means would be a computer readable medium, e.g., diskette, CD, etc., on which the information has been recorded.

What is claimed is:

1. A method for determining responsiveness of a subject to treatment with a Type I interferon (IFN) comprising detecting, in a sample from a subject, the mRNA or protein expression of one or more:
a) IFN-stimulated genes regulated by Signal Transducer and Activator of Transcription 2 (STAT2),
b) IFN-stimulated genes regulated by STAT2 and Nuclear factor-kappa-B (NF-κB),
c) IFN-stimulated genes regulated by STAT1/1, STAT1/3 OR STAT3/3 dimers, or
d) IFN-stimulated genes regulated by STAT1/1, STAT1/3 OR STAT3/3 dimers and NF-κB,
wherein the sample is obtained from the subject following administration of a Type I IFN or the sample is treated with a Type I IFN in vitro prior to detecting mRNA or protein expression, and wherein said mRNA or protein expression is indicative of the responsiveness of the subject to treatment with the Type I IFN.

2. The method of claim 1, wherein the IFN-stimulated genes regulated by STAT2 are selected from the group of Dhx58, Eif2ak2, IFIl6, IFI6, Irf2, Mx1, OAS1, PLSCR1, Pm1, RARRES3, Rsad2, Socs1, Stat2, Tap1, Tlr2, Tlr3 and Tnfsf10.

3. The method of claim 1, wherein the IFN-stimulated genes regulated by STAT2 and NF-κB are selected from the group of ANGPT2, Ccl5, Ddx58, Gbp1, IFIH1, Ifnb1, Il6, Irf5, Irf7, Isg20, IRF9 and Stat1.

4. The method of claim 1, wherein the IFN-stimulated genes regulated by STAT1/1, STAT1/3 or STAT3/3 dimers is selected from the group of Casp4, Tnfrsf10b, VEGFC and VISA.

5. The method of claim 1, wherein the IFN-stimulated genes regulated by STAT1/1, STAT1/3 or STAT3/3 dimers and NF-κB are selected from the group of Gadd45b, Gadd45g, HIF3A, Irf1, Myd88 and XAF1.

6. A method for testing the propensity of a test compound to induce a Type 1 interferon (IFN) response in a test subject comprising:
a) administering an effective amount of a test compound to a test subject or sample from the test subject; and
b) determining changes in mRNA or protein expression of one or more:
i) IFN-stimulated genes regulated by Signal Transducer and Activator of Transcription 2 (STAT2);
ii) IFN-stimulated genes regulated by STAT2 and Nuclear factor-kappa-B (NF-κB);
iii) IFN-stimulated genes regulated by STAT1/1, STAT1/3 OR STAT3/3 dimers; or
iv) IFN-stimulated genes regulated by STAT1/1, STAT1/3 OR STAT3/3 dimers and NF-κB,
wherein an increase in mRNA or protein expression is indicative of the propensity of the test compound to induce a Type I interferon response.

7. The method of claim 6, wherein the IFN-stimulated genes regulated by STAT2 are selected from the group of Dhx58, Eif2ak2, IFI16, IFI6, Irf2, Mx1, OAS1, PLSCR1, Pm1, RARRES3, Rsad2, Socs1, Stat2, Tap1, Tlr2, Tlr3 and Tnfsf10.

8. The method of claim 6, wherein the IFN-stimulated genes regulated by STAT2 and NF-κB are selected from the group of ANGPT2, Ccl5, Ddx58, Gbp1, IFIH1, Ifnb1, Il6, Irf5, Irf7, Isg20, IRF9 and Stat1.

9. The method of claim 6, wherein the IFN-stimulated genes regulated by STAT1/1, STAT1/3 or STAT3/3 dimers are selected from the group of Casp4, Tnfrsf10b, VEGFC and VISA.

10. The method of claim 6, wherein the IFN-stimulated genes regulated by STAT1/1, STAT1/3 or STAT3/3 dimers and NF-κB are selected from the group of Gadd45b, Gadd45g, HIF3A, Irf1, Myd88 and XAF1.

11. A kit comprising reagents for measuring expression of:
a) Dhx58, Eif2ak2, IFI16, IF16, Irf2, Mx1, OAS1, PLSCR1, Pm1, RARRES3, Rsad2, Socs1, Stat2, Tap1, Tlr2, Tlr3 and Tnfsf10,
b) ANGPT2, Ccl5, Ddx58, Gbp1, IFIH1, Ifnb1, Il6, Irf5, Irf7, Isg20, IRF9 and Stat1,
c) Casp4, Tnfrsf10b, VEGFC and VISA, or
d) Gadd45b, Gadd45g, HIF3A, Irf1, Myd88 and XAF1.
